(19) United States
(12) Patent Application Publication
(10) Pub. No.: US 2023/0329894 A1
(43) Pub. Date: Oct. 19, 2023

(54) Disposable Urinal Bag and Methods Associated Therewith

(71) Applicant: Robert L. Ballard, Birmingham, AL (US)

(72) Inventor: Robert L. Ballard, Birmingham, AL (US)

(21) Appl. No.: 17/720,075

(22) Filed: Apr. 13, 2022

(51) Int. Cl. A61F 5/44; A61B 10/00; A61F 5/453; A61F 5/455; A61F 5/441

(52) U.S. Cl. CPC A61F 5/4407; A61B 10/007; A61F 5/453; A61F 5/4556; A61F 5/441; A61F 5/4401; A61B 2010/0006; A61F 2005/4402

(57) ABSTRACT

A sanitary hand-held disposable portable urinal device is well suited for male use. In an embodiment, this easy to use device has a leak and splash resistant flexible, sealable opening that is attached to two flexible transparent plastic bags. In an embodiment, bags are nested inside of each other and are joined together to provide entry only to the inner bag. The bags are odor and vapor sealable and made of a hydrophobic polymer that may be biodegradable. In the space between the two bags a dry gel polymer is present. After the urine is collected in the inner bag the top of the bag is sealed. The contents of the inner bag are then exposed to the space containing the gel through a water soluble, or mechanical means. After use, the disposable urinal may be discarded.

### FIELD OF THE INVENTION

The present invention relates to a device and associated methods that can be used to provide an option for urination and the easy disposal of urine. The device comprises a disposable, portable urinal system for holding urine of a user during and after urination.

### BACKGROUND OF THE INVENTION

The emergence of the COVID-19 pandemic has closed many public and private enterprises/businesses. Often the ones that have remained open have either restricted their hours or restricted their uses. One enterprise that has greatly restricted its availability during the pandemic has been public and business lavatories (i.e., bathrooms). Moreover, even if the bathrooms are/were open, there are instances where a person who urinates may not be able to arrive at said location in a timely manner and may have to urinate prior to their arrival at said location. This may occur while someone is sitting in his/her car in a traffic jam or on a highway where the nearest exit is miles away. The person who needs to urinate may be camping in very cold weather and not want to leave the warmth of his/her tent in the middle of the night to go to the public restroom. There are also instances where the public restroom might be so dirty that the user finds it disgusting to use the restroom and wants a less repulsive means of relieving themselves.

Unfortunately, people do not have to use the bathrooms any less but their relative scarcity during the pandemic has forced people to either remain incredibly uncomfortable due to the need to urinate or forced people to urinate in locations that are not suitable and/or ideal locations.

There exist a number of disposable urinal devices that allow a user to urinate when not at a bathroom. However, these devices tend to suffer from a number of drawbacks. The first drawback is that most of these devices tend to leave the urine in the liquid form, which can be easily spilled, or the device punctured leading to urine spillage, which potentially causes odors that are not easily removed or dissipated. Another drawback is that the disposal of urine when in its liquid form can lead to the embarrassment of the person who urinates, when that person attempts to dispose of the urine. That person may not be able to conceal the urine when disposing of it, particularly because the urine is not only a liquid, but is often a bright yellow in color.

Current disposal devices that are available are plastic specimen type jars or plastic containers similar to milk jugs that may or may not contain a handle. These disposal devices may come with tops but the tops may be difficult to secure so that spillage is a potential problem. These devices are designed to leave the urine in its liquid form. There also exist bags that the manufacturers claim to be rip or puncture proof that can be used. There is a continuing need for a sanitary, disposable urinal system that is comfortable, easy to use, does not leak, is not foul smelling and is easy to store prior to use and prior to disposal. Unfortunately, all of the currently available devices do little to deal with the above enumerated problems.

It was with the above problems and drawbacks in mind that the present invention was developed.

### BRIEF SUMMARY OF THE INVENTION

The present invention relates to a device that allows a user to urinate in it wherein said device can be easily and non-conspicuously discarded. In an embodiment, the present invention relates to a device that is a disposable, portable urinal system for holding urine of a user during and after urination, which is designed so as to prevent or inhibit spillage after the user urinates in it. In an embodiment, the present invention relates to a device where a user urinates in it and the device contains a substance that causes the urine to ultimately change from a liquid to a gel, semi-solid, or solid. In an embodiment, the device may contain a color change agent that allows the urine to change from a yellow color to another color, including the color blue, green, pink, brown, black, red, orange, or some other color. In an embodiment, the device may actually be of a color that disguises the yellow color of urine.

### BRIEF DESCRIPTION OF THE SEVERAL VIEWS OF THE DRAWING

FIG. 1 shows a front view of an embodiment of the present invention wherein the string can be pulled at the bottom of the device and a removable funnel is present.

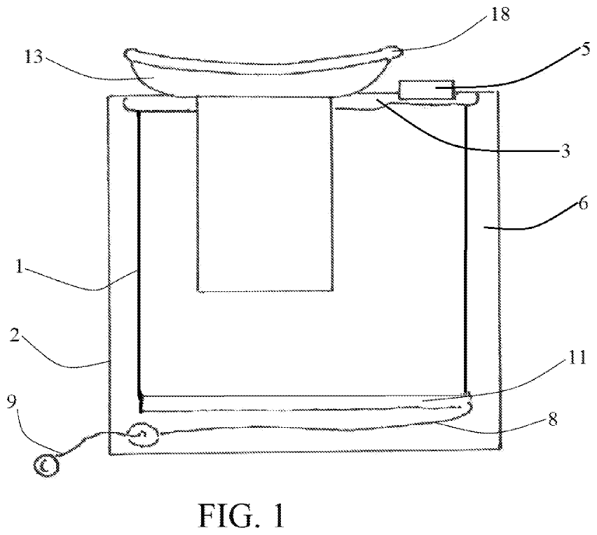

FIG. 2 shows a front perspective view of an embodiment of the present invention wherein the string is operationally connected to both the slide and the removable stitch and the polymer gel is present.

FIG. 3 shows a side perspective view of an embodiment of the present invention wherein the string is operationally connected to the removable stitch and the slide and the removable stitch is mainly intact.

FIG. 4 shows a perspective view of an embodiment of the invention wherein the removable stitch is largely unzipped and the slide closes the first outer bag.

FIG. 5 shows a front view of an embodiment of the invention wherein a capsule (pod) is present in the first outer bag.

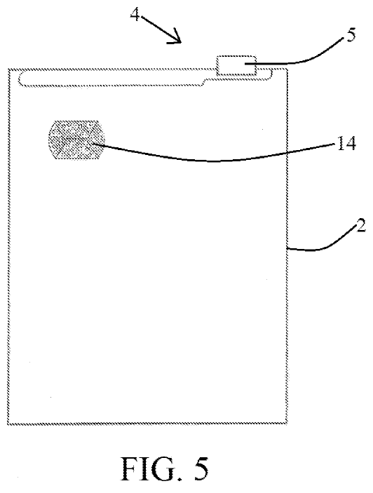

FIG. 6 shows a front view image of a kit comprising many of the components of the present invention.

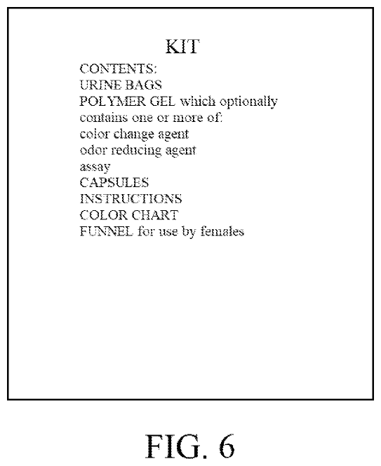

### DETAILED DESCRIPTION OF THE INVENTION

The present invention relates to a device that allows a user to urinate in it wherein said device can be easily and non-conspicuously discarded. In an embodiment, the present invention relates to a device where a user urinates in it and the device contains a substance that causes the urine to ultimately change from a liquid to a gel, semi-solid, or solid. In an embodiment, the device may contain a color change agent that allows the urine to change from a yellow color to another color, including the color blue, green, pink, brown, black, red, orange, or some other color.

In an embodiment, a smaller thin second inner bag 1 is positioned inside a larger first outer bag 2. The larger first outer bag 2 in an embodiment is equipped with a sealable opening that comprises a zip lock closure 3 and sealing device 4, the sealing device 4 being a slide 5 that causes the zip lock closure 3 to either open or close depending on the position of the slide. When the slide 5 is moved in one direction, the slide 5 causes the zip lock closure 3 to close thereby closing the first outer bag 2. When the slider is moved in the opposite direction, the slide 5 causes the zip lock closure 3 to open thereby opening the first outer bag 2. In the sealed space 6 that occurs outside of the second inner bag and inside of the first outer bag a quantity of a polymer gel 7 is present. This polymer gel 7 is comprised of a substance that absorbs liquid to an extent that the liquid becomes solid or at least semi-solid in form. Sufficient polymer gel 7 is present so as to absorb up to 400 ml of liquid. In an embodiment, the polymer gel 7 can absorb up to 70 times its weight in liquid and when the polymer gel 7 absorbs the liquid, a resultant solid or semi-solid material is formed.

In an embodiment, the polymer gel substance that may cause the liquid to be absorbed and to change the liquid to a solid or semi-solid may be one or more of sodium polyacrylate polymers, purified skin gelatin, bentonite clay, silica gel or silica dioxide, paper, pine or sawdust, or wheat or other plant products. In an embodiment, more than one of these substances can be used in combination.

In an embodiment, the polymer gel may optionally further comprise a color change agent that allows the change of urine from yellow to some other color. The color change agent should be selected so as to change colors in the pH range of urine. Generally, the pH range should be selected so as to effectuate a color change in the pH range of 4.5 to 8.0.

In an embodiment, the color change agent may be one or more of methylene blue, phenazopyridine, ethoxazene, rifampin, betalain, betanin, isobetanin, probetanin, neobetanin, phenolphthalein in combination with an alkaline substance such as potassium hydroxide, food dye and color blue number 1, 1-dopa, melanin, phenacetin, or phenol, or a combination of albumin, fibrin, emulsified lipids, cholesterol, and triglycerides, which will turn urine cloudy milky white. In an embodiment, there may be dyes or chromophores that may be added to the absorbing substance that have a high degree of conjugation that readily absorb at wavelengths in the visible region of the electromagnetic spectrum that when exposed to light allow the urine to change colors.

In an embodiment, the device may also comprise an agent that allows one to mask or eliminate the smell of urine. For example, the device may further comprise an oxidizing agent such as hydrogen peroxides that creates uric acid crystals that reduces the smell of the urine. The device may alternatively and/or additionally contain enzymes that are used to break down the compounds that contribute to the smell of urine. Other compounds or products that may be added to reduce or eliminate urine smells include urine-smell reducing bacteria, perfumes, surfactants, chlorine bleaches, charcoals, alkyl or aryl ammonium chlorides, fragrances, and/or metal oxide minerals comprising sodium, potassium, calcium, aluminum, iron, and/or silicon.

In an embodiment, the composition that has the polymer gel may also comprise an assay detecting agent that allows one to detect certain metabolites that are present in the urine. For example, the assay detecting agent may be a compound that detects blood (to see if there is blood in the urine), detects sugars (for diabetes), detects bacteria like Escherichia coli (for urinary tract infections), detects for kidney diseases, or has other enzymes that can detect other agents/metabolites that may be present in urine when a user is not normal. For example, the composition may comprise glucose oxidase in combination with horseradish peroxidase in a buffer such as sodium acetate that allows one to see a color change when glucose is present in the urine. The amount of glucose present affects the intensity of the color change so that one may be able to quantify the amount of glucose present in the urine. Similarly, other enzymatic reactions can be used to detect other metabolites that may be present in urine.

In an alternate embodiment, the first outer bag 2 may be either opaque or transparent or partially transparent. If the outer bag is opaque, it is of sufficient opaqueness that the color of the urine cannot be seen when an observer looks at the outside of the first outer bag 2. If the first outer bag 2 is transparent or at least partially transparent, the first outer bag 2 may be of a color that allows one to disguise the urine contained therein. For example, the first outer bag 2 may be blue in color, which would lead an observer to believe that the user has a green solid or semi-solid phase contained in the system (apparatus or device). Similarly, if the first outer bag 2 is red in color, the observer would believe an orange solid or semi-solid is contained therein.

In an embodiment, the polymer gel, which optionally contains a color change agent, an odor eliminating substance, and/or an appropriate assay detecting component may be present in a capsule 14 (similar to pods used in washing machines or dishwashers) wherein the polymer gel and the other optional components are released upon the pod getting “wet”. The capsule 14 material may be comprised of a polyvinyl alcohol (PVA) film, which can be mechanically or chemically ruptured. The capsule 14 can be chemically ruptured when it is exposed to wetness because the polyvinyl alcohol film is at least partially soluble in aqueous solutions (like urine). It can be mechanically ruptured because the PVA film is of a thickness that allows the capsule 14 to be broken open upon the exertion of sufficient force to rupture the film to release the contents. Thus, a user can rupture the capsule 14 by supplying sufficient pressure to the capsule, and/or additionally rupture the capsule by exposing it to a fluid (such as urine).

In an embodiment, the capsule 14 may take the place of the second inner bag 1. It should be understood that the capsule 14 may be considerably smaller than the first outer bag 2. Because the polymer gel is able to absorb up to 70 times its weight, it should be understood that less than 6 grams of polymer gel can be used, meaning that a smaller second inner bag 1 can be less than 10 cc's in size in the 400 cc first outer bag 2.

In an embodiment, the first outer bag 2 and the second inner bag 1 are made of low density polyethylene resin (polythene). The low density polyethylene resin may be made by blown film extrusion. The polyethylene resin may have a color dye added to it so that it attains a desired color. In an embodiment, the plastic may be a better biodegradable plastic made from glycerin and gelatin. The alternative biodegradable plastic may also have a dye added to it to attain the ideal color. The bags should be liquid and odor impenetrable. The first outer bag should have an opening on one side and the second inner bag should have an opening on a first end (top) and an opposite second end (bottom) of the second inner bag. The opposite second end (bottom) should have a removable stitch applied to it to seal the bottom of the bag wherein the removable stitch comprises a single stitch on a first side of the bag intertwined with a double stitch on the opposite second side of the bag. The removable stitch is applied in a manner that allows the removable stitch to be removed by pulling on an end of the thread on the single stitch.

In an embodiment, the bags are designed so that male genitalia fit easily in the bag. Generally, the second inner bag should be of a size that is large enough to easily contain up to 400 ml of liquid. In an embodiment, the second inner bag should be of a size wherein 400 ml of liquid occupies less than half the volume of the bag. Accordingly, in one embodiment, the second inner bag should be approximately 1 liter in volume. The first outer bag should be of a volume size that allows the second inner bag to fit in the interior of the first outer bag. In an embodiment, the first outer bag should be approximately 1.2 liters in volume. In an embodiment, the first outer bag and the second inner bag may be at least partially fused together on either one side or the other side of the exterior of the second inner bag and the interior of the first outer bag. In a variation, the first outer bag and the second interior bag are fused near the openings of the bags. The fusing may take place by the addition of a glue or the fusing may take place during the manufacture (e.g., during the extrusion process) of the bags simply by allowing the polyethylene to fuse together prior to the polyethylene changing state to its final plastic state. The fusing of the bags should make sure that the top openings are congruent with each other. By congruent, it is meant that the openings are positioned so that the male genitalia can be positioned inside both openings of the first outer bag and the second inner bag simultaneously when the user urinates allowing the user to urinate into the interior of the second inner bag.

In an embodiment, either of the first outer bag or the second inner bag has a piece of structural foam 17 associated with either bag on the interior that gives some structural rigidity to the bags yet still allows the first outer bag to be closed by means of the zip lock closure and slide. The structural foam 17 may be similar to a gusset that allows the shape of the opening of the first outer bag to remain substantially the same. The structural foam 17 may be made of a styrene based polymer (e.g., polystyrene) or alternatively, the structural rigidity may be obtained by using a poly lactic acid (or polylactate), Bagasse (the pulpy fibrous material made from sugarcane or sorghum, mineral filled polypropylene, polycoated paper, or a thin strip of a malleable metal (such as aluminum). It should be understood that if a polymer is to be used, the polymer may be made by a monomer or a copolymer. Ideally, the structural rigidity is such that it does not cause any discomfort to the user, but is sufficiently rigid so as to maintain its shape upon stress, yet sufficiently malleable so that the first outer bag can be easily closed at the sealable opening.

In an embodiment, the first outer bag has a zip lock closure with a slide attached at the opening of the first outer bag. This zip lock closure with slide allows the opening of the first outer bag to be closed when the slide is moved to a position that closes the zip lock closure. In an alternate embodiment, the first outer bag can be closed by a snap fit closure wherein one side of the bag contains a female snap fit that runs the length of the opening and the corresponding other side of the bag contains a male snap fit that runs the length of the opening that allows the male snap fit to fit into the corresponding female snap fit thereby closing the opening of the first outer bag.

In an embodiment, the slide 5 associated with the zip lock closure 3 on the first outer bag 2 is operationally connected to a string 8 on a first end 9 of the string. The second end 10 of the string 8 is operationally connected to a string sealed removable stitch 11 that is present on the bottom of the second inner bag 1 (the side opposite the opening of the second inner bag 1). The removable stitch 11 may comprise a combination of stitches that has a single stitch on one side of the bag and a double stitch on the other side of the second inner bag so that the removable stitch 11 is simply removed by pulling on the single stitch (this is a stitch that is similar to the stitch that is used on, for example, pet food bags or big rice bags that allows one to open the bag by pulling the thread on the single stitch side). Because the string 8 is operationally connected to the slide 5 on a first end 9 of the string 8 and operationally connected to the removable stitch at the bottom of the second inner bag 1 by the second end 10 of the string 8, when the slide 5 is moved to a position that closes the first outer bag 2, the attached string 8 also simultaneously opens the removable stitch 11, thereby also opening the bottom of the second inner bag 1. The string 8, in an embodiment, is present in the sealed space 6 that occurs between the inside of the first outer bag 2 and outside of the second inner bag 1. When the bottom of the second inner bag 1 is opened, the contents therein (usually urine) is released to the sealed space 6 that occurs outside the second inner bag 1 and inside the first outer bag 2. The urine will contact the polymer gel 7 and turn to a solid or semi-solid state. If a color change agent is present, the urine will change to the appropriate color effected by the color change agent.

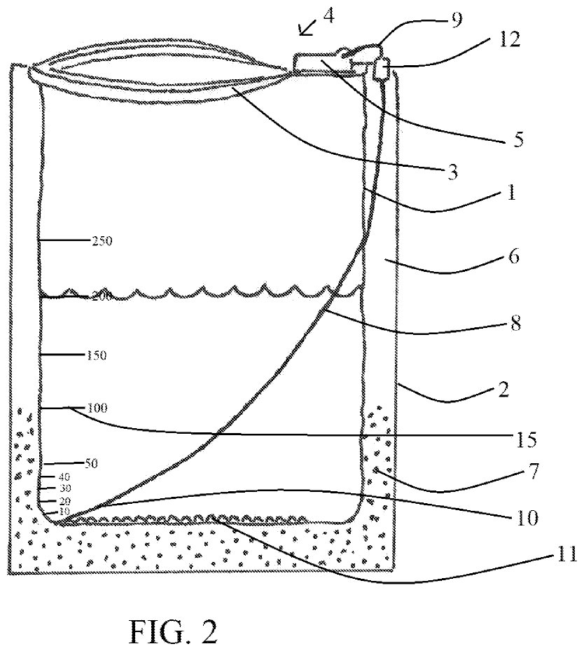

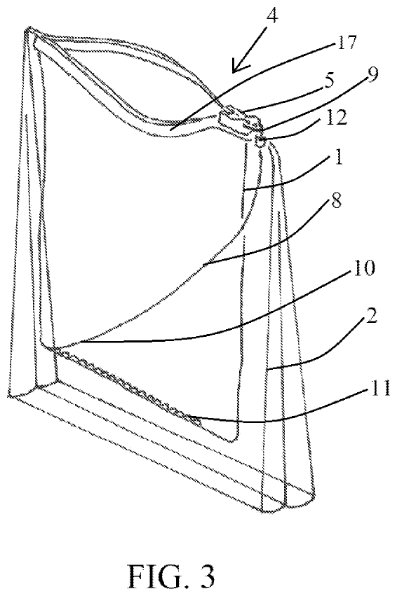

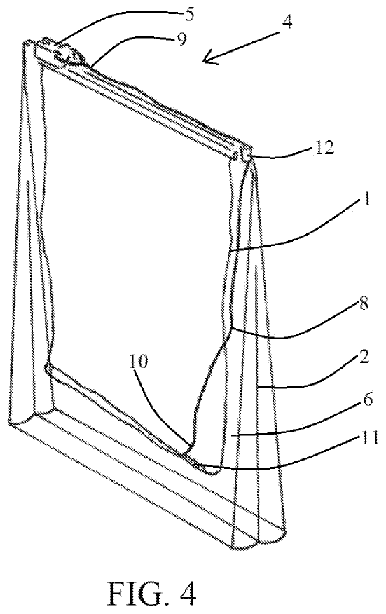

In an embodiment, the string/thread 8 when pulled by the slide 5 is positioned through a short cylindrical tube 12 that is affixed at a position in the first outer bag 2 so that when the string 8 is pulled the removable stitch 11 is unzipped. This short cylindrical tube 12 is rigid and because it remains affixed in one position, the short cylindrical tube 12 serves the purpose of allowing a pulling force to be placed upon the string when the slide 5 is closed. This pulling force allows the removable stitch 11 to be unzipped. See FIGS. 2-4. As shown in FIG. 3, the removable stitch 11 is mainly zipped because the slide has not been moved to close the opening at the tops of the first outer bag 2 and the second inner bag 1. As shown in FIG. 4, the removable stich 11 is largely unzipped because the slide 5 is in a position that causes the removable stitch 11 to be unzipped. Because the string is a fixed length, the short cylindrical tube 12 serves the purpose of allowing the string to unzip the removable stitch 11 when the slide is moved to a position that closes the opening in the first outer bag 2 and the second inner bag 1. In an embodiment, when the bottom of the second outer bag 1 is opened and the urine that is/was present therein is allowed to contact the polymer gel 7, the user may shake the system (device or apparatus) to allow the urine to appropriately contact the polymer gel 7, thereby allowing the urine 19 (shown in FIG. 2) to effectuate a change from a liquid to a solid or semi-solid.

In a variation, the string may be operationally connected on a second end 10 of the string to the removable stitch 11 but not operationally connected at a first end 9 of the string 8 to the slide 5. The string 8 may be present at the slide 5 in this embodiment and require the user to pull on the first end 9 of the string 8 for the user to manually open the removable stitch 11 to open the bottom of the second inner bag 1 thereby releasing the contents contained in the second inner bag 1 to the sealed space 6 thereby allowing the contents (usually urine) to contact the sealed space 6 that occurs outside the second inner bag 1 but inside the first outer bag 2. The urine may then contact the polymer gel 7 thereby changing in state from a liquid to solid or semi-solid state.

In an embodiment, the second inner bag may not have a sealed removable stitch associated with it. In this embodiment, the second inner bag is positioned inside the first outer bag. The openings of the two bags may be congruent and the user urinates into the openings and the urine remains in the interior of the second inner bag. The second inner bag may have the polymer gel already in the interior of the second inner bag so that when the user urinates the flowing urine immediately contacts the polymer gel and changes upon contact from a liquid state to a gel, semi-solid or solid state. Alternatively, the user may optionally have the option of adding the polymer gel to the interior of the second bag after the user urinates such as might occur if the device of the present invention is part of a kit (explained in more detail below).

In an embodiment, the bottom of the second inner bag 1 may optionally be opened by string and tape wherein the tape is positioned and affixed to the outside of the first outer bag 2 and the tape is attached to the string so that when the tape is pulled off the outside of the first outer bag 2, the tape pulls the string that is attached to it. The string on one end is attached to the tape, and the string is passed through a plastic plug that is present in the outer bag and the other end of the string is present at the bottom of the second inner bag 1 wherein the string forms the seal at the bottom of the second inner bag 1. When the tape is removed, the attached string is pulled in a manner that opens a removable stich at the bottom of the second inner bag 1 thereby opening the bottom of the second inner bag 1 and releasing the contents of the second inner bag 1 to the inside of the first outer bag 2.

In a variation, the tape may alternatively be positioned so that it is at the bottom of the second inner bag 1 in the sealed space 6 with string attached to the tape. The string may exit through a passageway in first outer bag 2 so that one end of the string is present outside of the device. In a variation, the string passes through an impermeable or partially permeable plug such as silastic, rubber or cork. When the end of the string is pulled the tape is in turn pulled off of the bottom of second inner bag 1 allowing the contents of second inner bag 1 to empty into the sealed space 6. The sealed space may optionally have the polymer gel 7 present (and any accompanying additions to the polymer gel as discussed herein) in the sealed space 6.

In an embodiment, the second inner bag may not have a removable stitch associated with it but alternatively have a sealant associated with the bottom of the second inner bag that will remain sealed permanently when no liquid contacts it but upon contact by a liquid will remain sealed for a period of time when contacted by a liquid but eventually will be degraded allowing the bottom of the second bag to open. Ideally, the sealant will remain in place and the bottom of the second inner bag will remain sealed for a period of between 30 seconds to one minute after contact by a liquid (e.g., urine). After this duration of time, the liquid urine will be released from the space that is on the interior of the second inner bag to the sealed space that is exterior the second inner bag but interior to the first outer bag. The sealed space may have the polymer gel present so that when the urine undergoes passage to the sealed space it undergoes the transition from liquid urine to the gel, semi-solid, or solid composition.

In an embodiment, the second inner bag may have graduated markings 15 printed on the outside of the second inner bag that allows a user (or someone else) to ascertain the volume of the contents in the inner bag. In an embodiment, the device may contain graduated markings 15 so that the first hundred milliliter volume of the contents can be precisely determined. The graduated markings 15 may be printed in five milliliter increments (or even less) that allows a user to determine rather precisely and accurately the amount of the contents (e.g., urine) present. In a variation, the markings may be greater (for example, the increment amounts may be in 25 ml increments) when the volume is greater than 100 ml.

In an embodiment, the system of the present invention may come as a kit wherein the kit contains a first outer bag and a second inner bag that are combined together. The sealable opening devices and the string and the polymer gel (which optionally contains a color change agent) may come as separate components of the kit. The kit might potentially come with a plurality of individual units of packaged polymer gel with the optional components discussed above that allows the user to add the polymer gel as needed to the sealed space that is inside the first outer bag but outside the second inner bag. The kit may come with five or more bags and five or more individually packaged polymer gel units. The kit may also come with a color change agent that can be added to the individually packaged polymer gel units. The kit may come with one or more capsules 14 as discussed above and the capsules 14 may have components contained therein that allow for the testing and quantification of metabolites. The kit may come with instructions and a color chart that allow the user to quantitatively test for these metabolites. The kit may be of a size that allows it to easily fit inside the glove box of an automobile so that a user will have it with him when traveling.

Although the urinal system has been disclosed with respect to use by males, it should be understood that the urinal system can also be adapted to be used by females. Accordingly, in an embodiment, the present invention may be adapted for female use by the addition of a conical funnel 13 that is partially inserted into the second inner bag. The funnel 13 may be constructed with a skeleton of plastic, or a biodegradable material such as card-board that may optionally be lined with a hydrophobic material, such as plastic, or a biodegradable material such as waxed paper.

The funnel 13 may be reversibly, or irreversibly attached to the rim of the device, or to the inner lining of the second inner bag using an adhesive, hook and loop fastener, or other mechanical attachment. In an embodiment, the rim of the proximal larger portion of the funnel 13 has a piece of structural foam 18 that gives some structural rigidity and skin protection to the perineum. In an embodiment, the funnel 13 may not be exactly conical in shape but the proximal larger portion of the funnel 13 may be shaped in a manner that directly accommodates the shape of the user or the proximal larger portion although it has some structural rigidity may have sufficient elasticity so that it can be bent to accommodate the shape of the user.

In an embodiment, the structural foam may be made of a styrene based polymer (e.g., polystyrene) or alternatively, the structural rigidity may be obtained by using a polylactic acid (for example, polylactate, Bagasse (the fibrous material made from sugarcane or sorghum), mineral filled polypropylene, polycoated paper, or a thin strip of malleable metal (such as aluminum)). It should be understood that if a polymer is to be used, the polymer may be made by a monomer, or copolymer. Ideally, the structural rigidity is such that it does not cause any discomfort to the user, but is sufficiently rigid so as to maintain its shape upon stress, but sufficiently malleable so that the funnel 13 may be collapsed. Following urination, the funnel 13 could be discarded independently, or collapsed into the lumen of second inner bag, prior to sealing the device.

The device of the present invention may also have other modifications that can be used for example, on a user that has an ostomy by the user or by medical personnel. Generally, an ostomy is a surgically generated hole generated and used by a surgeon, for example, in the abdomen that allows urine to pass through said hole. The device may be adapted by having an adapter that allows the ostomy to pass urine to the device by an adapter (such as a cannula) wherein the adapter is designed to allow passage of the urine to the second inner bag. The opening of the second inner bag may be adapted so as to ideally fit the adaptor. For example, if a cannula is used, the sealable opening at the top of the second inner bag or at the top of the first outer bag may be of a size so as to directly accommodate the diameter of the cannula.

In an embodiment, the device of the present invention may also be used to collect fluids from any of ascites, pleural effusions, abscesses, colostomies, hydronephrosis, incontinence, emises, or as a means of collecting any other bodily fluid that may build up. The device of the present invention may be particularly useful after surgery where fluid collection may be required.

Thus, in an embodiment, the present invention relates to a disposable, portable urinal system for holding urine of a user during and after urination, comprising: a first outer bag and a second inner bag, the second inner bag being smaller than the first outer bag and disposed within the first outer bag, wherein both the first outer bag and the second inner bag comprise:

substantially a liquid and odor impenetrable material, the first outer bag and the second inner bag comprising openings so that the openings are positioned so as to be congruent with each other, the first outer bag having at least one sealable opening wherein said system is structured and arranged to closely fit male genitalia.

In a variation, the system further comprises a polymer gel that absorbs liquids. The polymer gel may be disposed in a space that is outside the second inner bag but inside the first outer bag. The polymer gel may alternatively be inside the second inner bag or may be contained in an individually packaged package that can be added to the sealed space or the space that is on the interior of the second inner bag.

In an embodiment, the polymer gel is one or more members selected from the group consisting of sodium polyacrylate polymers, purified skin gelatin, bentonite clay, silica gel, silica dioxide, paper, pine sawdust, and wheat.

In an embodiment, the sealable opening is comprised of a zip lock closure. The zip lock closure may comprise a slide. The zip lock closure may be a snap fit zip lock closure.

In a variation, the polymer gel may further comprise a color change agent. The color change agent may be one or more members selected from the group consisting of methylene blue, phenazopyridine, ethoxazene, rifampin, betalain, betanin, isobetanin, probetanin, neobetanin, phenolphthalein in combination with potassium hydroxide, food dye color blue number 1, 1-dopa, melanin, phenacetin, phenol, and a combination of albumin, fibrin, emulsified lipids, cholesterol, and triglycerides.

In an embodiment, either or both of the first outer bag and second inner bag are transparent and/or are colored. Alternatively, either or both of the first outer bag and second inner bag are opaque.

In an embodiment, at least a part of the first outer bag and the second inner bag are fused together.

In an embodiment, the polymer gel is able to absorb liquid up to 70 times its weight. In an embodiment, the size of the inner bag is about 1 liter and the amount of polymer gel is enough to absorb up to 400 ml of urine.

In an embodiment, the second inner bag comprises graduated markings 15 allowing a user to ascertain a volume of urine. In an embodiment, the graduated markings 15 are present at 25 ml increments. In a variation, the graduated markings 15 are present at 50 ml increments. In a variation, the graduated markings 15 are present at 100 ml increments.

In an embodiment, the sealable opening further comprises a piece of structural foam that allows the openings to remain rigid when open or closed. In a variation, the structural foam comprises polystyrene.

In an embodiment, the second inner bag further comprises a removable stitch. The removable stitch may be a stitch that comprises a single stitch on one side of the inner second bag and a double stitch on the other side of the second inner bag. In an embodiment, the removable stitch is removable by means of a string that is operationally attached at a first end of the string to the slide and operationally connected to the removable stitch at the second opposite end of the string.

In an embodiment, the second inner bag further comprises a sealant. The sealant may be a sealant that degrades upon contact with a liquid. In an embodiment, when the sealant degrades an opening at the bottom of the second inner bag occurs.

In an embodiment, the system comes as a kit.

It is contemplated that any of the features of the present invention that are described herein can be combined with any other feature that is described herein even if they are not described together (as long as they are compatible). When ranges are discussed every real number that is present in that range is contemplated as an end point for a subrange within that range. In any event, the invention is best defined by the below claims.

I claim:

1. A disposable, portable urinal system for holding urine of a user during and after urination, comprising:
a first outer bag and a second inner bag, the second inner bag being smaller than the first outer bag and disposed within the first outer bag, wherein both the first outer bag and the second inner bag comprise:
substantially a liquid and odor impenetrable material, the first outer bag and the second inner bag comprising openings so that the openings are positioned so as to be congruent with each other, the first outer bag having at least one sealable opening wherein said system is structured and arranged to closely fit male or female genitalia.

2. The system of claim 1, further comprising a polymer gel that absorbs liquids.

3. The system of claim 2, wherein the polymer gel is disposed in a space that is outside the second inner bag but inside the first outer bag or alternatively, in the second inner bag.

4. The system of claim 2, wherein the polymer gel is one or more members selected from the group consisting of sodium polyacrylate polymers, purified skin gelatin, bentonite clay, silica gel, silica dioxide, paper, pine sawdust, and wheat.

5. The system of claim 4, wherein the sealable opening is comprised of a zip lock closure.

6. The system of claim 5, wherein the zip lock closure comprises a slide.

7. The system of claim 4, wherein the polymer gel further comprises one or more of one or more color change agent, one or more odor reducing agent, or one or more compounds used to assay for metabolites.

8. The system of claim 7, wherein the color change agent is one or more members selected from the group consisting of methylene blue, phenazopyridine, ethoxazene, rifampin, betalain, betanin, isobetanin, probetanin, neobetanin, phenolphthalein in combination with potassium hydroxide, food dye color blue number 1, 1-dopa, melanin, phenacetin, phenol, and a combination of albumin, fibrin, emulsified lipids, cholesterol, and triglycerides.

9. The system of claim 1, wherein either or both of the first outer bag and second inner bag are transparent and/or are colored.

10. The system of claim 1, wherein either or both of the first outer bag and second inner bag are opaque.

11. The system of claim 1, wherein at least a part of the first outer bag and the second inner bag are fused together.

12. The system of claim 4, wherein the polymer gel is able to absorb liquid up to 70 times its weight.

13. The system of claim 1, wherein the second inner bag comprises graduated markings allowing a user to ascertain a volume of urine.

14. The system of claim 1, wherein the sealable opening further comprises a piece of structural foam that allows the openings to remain rigid when open or closed and/or comprises a conical funnel that is adapted to fit in the sealable opening that is adapted to fit female genitalia.

15. The system of claim 14, wherein the structural foam comprises polystyrene.

16. The system of claim 6, wherein the second inner bag further comprises a removable stitch or the second inner bag is a capsule that is made of a polyvinyl alcohol film.

17. The system of claim 1, wherein the second inner bag further comprises a sealant.

18. The system of claim 16, wherein the removable stitch is removable by means of a string that is operationally attached at a first end of the string to the slide and operationally connected to the removable stitch at the second opposite end of the string.

19. The system of claim 17, wherein the sealant degrades upon contact with a liquid.

20. The system of claim 1, wherein the system comes as a kit.
